(19) United States
(12) Patent Application Publication
(10) Pub. No.: US 2011/0053161 A1
(43) Pub. Date: Mar. 3, 2011

(54) SEQUENTIAL SAMPLING FOR UNEXPECTED DAMAGE TO BT CORN FOR CORN ROOTWORM CONTROL

(75) Inventors: Bo Hong, North Mankato, MN (US); Elizabeth D. Owens, Urbandale, IA (US)

(73) Assignee: Pioneer Hi-Bred International, Inc., Johnston, IA (US)

(21) Appl. No.: 12/872,316

(22) Filed: Aug. 31, 2010

Related U.S. Application Data
(60) Provisional application No. 61/238,323, filed on Aug. 31, 2009.

(51) Int. Cl. C12Q 1/68; G01N 33/52

(52) U.S. Cl. 435/6; 435/7.92

(57) ABSTRACT

Methods are provided for the sequential sampling of pest resistant crop plants for determining resistance of a pest to a pesticidal activity of a pest resistant crop plant. The methods involve choosing a plant in a plot in an unbiased sampling manner. Trait expression is determined for each of the transgenic pest resistant plants to identify plants having pesticidal activity, and plants determined to have the pesticidal activity are a batch. Then the pest damage to the roots of the batch is rated with each batch having at least about five plants to determine the resistance of pests in the plot.

### CROSS-REFERENCE TO RELATED APPLICATIONS

This utility application claims the benefit U.S. Provisional Patent Application No. 61/238,323, filed Aug. 31, 2009, which is hereby incorporated herein in its entirety by reference.

### FIELD OF THE INVENTION

The present invention relates to methods for managing the development of resistant pests.

### BACKGROUND OF THE INVENTION

Insects, nematodes, and related arthropods annually destroy an estimated 15% of agricultural crops in the United States and even more than that in developing countries. Yearly, these pests cause over $100 billion dollars in crop damage in the U.S. alone. Corn rootworm (CRW) can result in yield losses of 8-16% of the total 11.8 million bushels of grain harvested (2004).

Some of this damage occurs in the soil when plant pathogens, insects and other such soil borne pests attack the seed after planting. In the production of corn, for example, much of the damage is caused by rootworms, insect pests that feed upon or otherwise damage the plant roots, and by cutworms, European corn borers, and other pests that feed upon or damage the above ground parts of the plant. General descriptions of the type and mechanisms of attack of pests on agricultural crops are provided by, e.g., Metcalf (1962), in Destructive and Useful Insects, 4th ed. (McGraw-Hill Book Co., NY); and Agrios (1988), in Plant Pathology, 3d ed. (Academic Press, NY).

In an ongoing seasonal battle, farmers must apply billions of gallons of synthetic pesticides to combat these pests. However, synthetic pesticides pose many problems. They are expensive, costing U.S. farmers alone almost $8 billion dollars per year. They force the emergence of insecticide-resistant pests, and they can harm the environment.

Because of concern about the impact of pesticides on public health and the health of the environment, significant efforts have been made to find ways to reduce the amount of chemical pesticides that are used. Recently, much of this effort has focused on the development of transgenic crops that are engineered to express insect toxicants derived from microorganisms. For example, since 2003, transgenic CRW-protected Bacillus thuringiensis (Bt) corn has been available to farmers (Monsanto Cry3Bb1) targeting the western corn rootworm (WCRW, Diabrotica virgifera virgifera, LeConte), northern corn rootworm (NCRW, D. barberi, Smith and Lawrence) and Mexican corn rootworm (MCRW, D. virgifera zeae, Krysan and Smith). Also, U.S. Pat. No. 5,877,012 to Estruch et al. discloses the cloning and expression of proteins from such organisms as Bacillus, Pseudomonas, Clavibacter and Rhizobium into plants to obtain transgenic plants with resistance to such pests as black cutworms, armyworms, several borers and other insect pests. Publication WO/EP97/07089 by Privalle et al. teaches the transformation of monocotyledons, such as corn, with a recombinant DNA sequence encoding peroxidase for the protection of the plant from feeding by corn borers, earworms and cutworms. Jansens et al. (1997) Crop Sci., 37(5): 1616-1624, reported the production of transgenic corn containing a gene encoding a crystalline protein from Bt that controlled both generations of European Corn Borer (ECB). U.S. Pat. Nos. 5,625,136 and 5,859,336 to Koziel et al. reported that the transformation of corn with a gene from Bt that encoded for a δ-endotoxin provided the transgenic corn with improved resistance to ECB. A comprehensive report of field trials of transgenic corn that expresses an insecticidal protein from Bacillus thuringiensis (Bt) has been provided by Armstrong et al., in Crop Science, 35(2):550-557 (1995).

An environmentally friendly approach to controlling pests is the use of pesticidal crystal proteins derived from the soil bacterium Bt, commonly referred to as “Cry proteins” or “Cry peptides.” The Cry proteins are globular protein molecules which accumulate as protoxins in crystalline form during late stage of the sporulation of Bt. After ingestion by the pest, the crystals are solubilized to release protoxins in the alkaline midgut environment of the larvae. Protoxins (˜130 kDa) are converted into toxic fragments (˜66 kDa N terminal region) by gut proteases. Many of these proteins are quite toxic to specific target insects, but harmless to plants and other non-targeted organisms. Some Cry proteins have been recombinantly expressed in crop plants to provide pest-resistant transgenic plants. Among those, Bt-transgenic cotton and corn have been widely cultivated.

A large number of Cry proteins have been isolated, characterized and classified based on amino acid sequence homology (Crickmore et al., 1998, Microbiol. Mol. Biol. Rev., 62: 807-813). This classification scheme provides a systematic mechanism for naming and categorizing newly discovered Cry proteins. The Cry1 classification is the best known and contains the highest number of cry genes which currently totals over 130. Specific, non-limiting examples of Bt Cry toxins of interest include the group consisting of Cry 1 (such as Cry1A, Cry1A(a), Cry1A(b), Cry1A(c), Cry1C, Cry1D, Cry1E, Cry1F), Cry 2 (such as Cry2A), Cry 3 (such as Cry3Bb), Cry 5, Cry 8 (see GenBank Accession Nos. CAD57542, CAD57543, see also U.S. patent application Ser. No. 10/746,914), Cry 9 (such as Cry9C) and Cry34/35, as well as functional fragments, chimeric or shuffled modifications, or other variants thereof. These insect toxins include, but are not limited to, the Cry toxins, including, for example, Cry1, Cry3, Cry5, Cry8, Cry9, Cry 34, and Cry 35.

In certain applications the plants produce more than one pesticidal activity, for example, via gene stacking. For example, DNA constructs in the plants of the embodiments may comprise any combination of stacked nucleotide sequences of interest in order to create plants with a desired trait. Alternatively, plants having different pesticidal activities may be planted in the same plot. For example, a mixture of transgenic seed may contain different modes of pesticidal action. See U.S. Publication No. 20080226753.

Corn rootworm (CRW) has four major species: western corn rootworm (WCRW D. virgifera virgifera, LeConte), northern corn rootworm (NCRW, Diabrotica barberi, Smith and Lawrence) and Mexican corn rootworm (MCRW D. virgifera zeae, Krysan and Smith) as mentioned previously, and Southern corn rootworm (D. undecimpunctata howardi, Barber). WCRW is the most prevalent rootworm target pest in the United States with NCRW second and the MCRW is limited to Texas. Cry 34 and Cry35 are effective against WCRW, NCRW and MCRW.

In the lifecycle of a CRW an adult female deposits eggs in a corn field during late summer; eggs overwinter and hatch in late-spring (late May-early June); larvae feed on corn roots for 3-4 weeks; mature into adult beetles which emerge from the soil in mid-July to feed on corn plants; mate and deposit eggs. Recently, deviations from the traditional lifestyle have occurred. One biotype of WCRW that is depositing its eggs in soybeans (and possibly other crop habitats) is now capable of causing significant injury to first-year corn (i.e., corn that has not systematically followed corn). This biotype is commonly called first-year corn rootworm or rotation-resistant corn rootworm. Another deviation has been seen in NCRW, especially in the northwestern region of the Corn Belt, where first-year corn may also be susceptible to rootworm injury when eggs remain in the soil for more than a year. In this situation, the eggs deposited in the plot remain dormant throughout the following year and then hatch the next year, when corn may again be planted in a two-year rotation cycle. Such rootworm activity is called extended diapause. Both of these deviations are examples of adaptations which lessen the effectiveness of crop rotation in pest management thus increasing the demand for other methods such as transgenic crops.

Further, most countries, including the United States, require extensive registration requirements when transgenic crops are used in order to minimize the development of resistant pests, and thereby extend the useful life of known biopesticides. One of the most common examples of a refuge is where in a given crop, 80% of the seed planted may contain a transgenic event which kills a target pest (such as CRW), but 20% of the seed must not contain that transgenic event. The goal of such a refuge strategy is prevent the target pests from developing resistance to the particular biopesticide produced by the transgenic crop. Because those target insects that reach maturity in the 80% transgenic area will likely be resistant to the biopesticide used there, the refuge permits adult CRW insects to develop that are not resistant to the biopesticide used in the transgenic seeds. As a result, the non-resistant insects breed with the resistant insects, and, because the resistance gene is typically recessive, eliminate much of the resistance in the next generation of insects. The problem with this refuge strategy is that in order to produce susceptible insects, some of the crop planted must be susceptible to the pest, thereby reducing yield.

As indicated above, one concern in planting transgenic pest resistant crop plants is that resistant CRW, or other pests will emerge. Another strategy for combating the development of resistance is to select a recombinant corn event which expresses high levels of the insecticidal protein.

Another strategy would be to combine a second CRW specific insecticidal protein in the form of a recombinant event in the same plant or in an adjacent plant, for example, another Cry protein or alternatively another insecticidal protein such as a recombinant acyl lipid hydrolase or insecticidal variant thereof. See, e.g., WO 01/49834. Preferably, the second toxin or toxin complex would have a different mode of action than the first toxin, and preferably, if receptors were involved in the toxicity of the insect to the recombinant protein, the receptors for each of the two or more insecticidal proteins in the same plant or an adjacent plant would be different so that if a change of function of a receptor or a loss of function of a receptor developed as the cause of resistance to the particular insecticidal protein, then it should not and likely would not affect the insecticidal activity of the remaining toxin which would be shown to bind to a receptor different from the receptor causing the loss of function of one of the two insecticidal proteins cloned into a plant. Accordingly, the first one or more transgenes and the second one or more transgenes are preferably insecticidal to the same target insect and bind without competition to different binding sites in the gut membranes of the target insect.

Still another strategy would combine a chemical pesticide with a pesticidal protein expressed in a transgenic plant. This could conceivably take the form of a chemical seed treatment of a recombinant seed which would allow for the dispersal into a zone around the root of a pesticidally controlling amount of a chemical pesticide which would protect root tissues from target pest infestation so long as the chemical persisted or the root tissue remained within the zone of pesticide dispersed into the soil.

Another alternative to the conventional forms of pesticide application is the treatment of plant seeds with pesticides. The use of fungicides or nematicides to protect seeds, young roots, and shoots from attack after planting and sprouting, and the use of low levels of insecticides for the protection of, for example, corn seed from wireworm, has been used for some time. Seed treatment with pesticides has the advantage of providing for the protection of the seeds, while minimizing the amount of pesticide required and limiting the amount of contact with the pesticide and the number of different field applications necessary to attain control of the pests in the field.

Other examples of the control of pests by applying insecticides directly to plant seed are provided in, for example, U.S. Pat. No. 5,696,144. In addition, U.S. Pat. No. 5,876,739 to Turnblad et al. and its parent, U.S. Pat. No. 5,849,320, disclose a method for controlling soil-borne insects which involves treating seeds with a coating containing one or more polymeric binders and an insecticide. This reference provides a list of insecticides that it identifies as candidates for use in this coating and also names a number of potential target insects.

Although recent developments in genetic engineering of plants have improved the ability to protect plants from pests without using chemical pesticides, and while such techniques have reduced the harmful effects of pesticides on the environment, numerous problems remain that limit the successful application of these methods under actual field conditions. One such problem is the threat insect resistance poses to the future use of Bt plant-incorporated protectants and Bt technology as a whole. Specific IRM strategies, such as the high dose/structured refuge strategy, mitigate insect resistance to specific Bt proteins produced in corn, cotton, and potatoes. However, such strategies need monitoring of pest populations in order to quickly detect possible pest resistance.

Monitoring is also necessitated because from a farmer/producer's perspective, it is highly desirable to have as small a refuge as possible; thus some farmers choose to eschew the refuge requirements, and others do not follow the size and/or placement requirements. These non-compliance issues result in either no refuge or less effective refuge, and a corresponding increased risk of the development of resistance pests.

Due to potential compliance problems, regulatory requirements, and also the importance of trait durability, strategies to insure successful management and monitoring have been developed. These are implemented through field reports of unexpected damage by the target pest and population testing and sampling. Field monitoring data is used to determine normal level of pest damage in a field from a pest such as rootworm. Once field guidelines are established, reports of pest damage are evaluated to determine if crop damage is higher than the expected level, i.e. unexpected level of damage, due to failure of the transgene encoding pesticidal activity or to some alternative factor. For example, current sampling to identify resistance to traits conferring resistance or tolerance to pests that damage maize roots includes choosing plants from a field exhibiting an unexpected level of damage, determining trait expression, rating roots and, if necessary, determining alternative causes of damage or lodging such as non-target pest insect species, weather, physical damage, planting errors and other factors. In cases where the unexpected level of damage cannot be accounted for by factors other than a potential resistant pest population, remedial actions to control the spread of resistance are implemented. These required local actions include additional measures to control the pest, such as pesticide applications and cultivation practices which are both costly and resource intensive for the grower. Ultimately, remedial action can result in loss of the use of pesticidal transgenic plants for control of the pest.

Methods for improving sampling for pests and the level of damage that they cause are therefore important and necessary to insure successful management of the pest and to promote durability of a resistance trait.

### BRIEF SUMMARY OF THE INVENTION

Embodiments of the invention therefore relate to methods of sequential sampling for determining resistance of a pest to a pesticidal activity of a transgenic pest resistant crop plant in a plot by choosing a plant in an unbiased sampling manner appropriate for said plot. Trait expression is determined for each of the transgenic pest resistant plants to identify plants having pesticidal activity, and plants determined to have the pesticidal activity are a batch. Then the pest damage to the roots of each of the plants in the batch is rated with each batch having at least about 5 and up to about 50 plants, per 100 acres in order to determine the resistance of pests in the plot.

Embodiments of the invention further relate to methods of sampling plots of maize to determine root damage by randomly selecting at least five plants per hundred acres from the field with no two selected plants within a set distance of each other. Each selected plant's root system is analyzed by determining the number of roots on the 3 latest fully developed nodes in each plant's root system and scoring the root mass based on an established scale where a set limit has been predetermined from experimental data. The plant score is either at, above or below the set limit. Then a composite score is calculated for the field based on the number of plants in the batch scoring at or above the set limit. In one embodiment of the invention, the set limit is two for a batch of five plants. In another embodiment, the set limit is four for a batch of five plants.

### DETAILED DESCRIPTION OF THE INVENTION

In the description that follows, a number of terms are used extensively. The following definitions are provided to facilitate understanding of the embodiments of the invention.

The article “a” and “an” are used herein to refer to one or more than one (i.e., to at least one) of the grammatical object of the article. By way of example, “an element” means one or more element. As used herein, the term “comprising” means “including but not limited to.”

As used herein, the terms “corn” or “maize” includes all plant varieties that can be bred with Zea mays, corn and maize, including wild maize species. In one embodiment, the disclosed methods are useful for monitoring resistance in a plot of pest resistant corn, where corn is systematically followed by corn (i.e., continuous corn). In another embodiment, the methods are useful for monitoring resistance in a plot of first-year pest resistant corn, that is, where corn is followed by another crop (e.g., soybeans), in a two-year rotation cycle. Other rotation cycles are also contemplated in the context of the invention, for example where corn is followed by multiple years of one or more other crops, so as to prevent resistance in other extended diapause pests that may develop over time.

As used herein, the term “creating or enhancing insect resistance” is intended to mean that the plant, which has been genetically modified in accordance with the methods of the present invention, has increased resistance to one or more insect pests relative to a plant having a similar genetic component with the exception of the genetic modification described herein.

By “crop plants” is intended a plant, purposely planted and harvested or utilized. Preferably, crop plants are monocotyledonous or dicotyledonous plants and are used for such purposes as, but not limited to, food, feed, fuel and combinations thereof. Crop plants include, but are not limited to, maize, sorghum, sunflower, soybean, wheat, alfalfa, rice, cotton, canola, barley, millet, and tomato. A particularly preferred monocotyledonous crop plant is maize.

As used herein, the term “pesticidal” is used to refer to a negative effect, such as but not limited to a toxic effect against a pest (e.g., CRW), and includes activity of either, or both, an externally supplied pesticide and/or an agent that is produced by the crop plants.

As used herein, the terms “pesticidal activity” and “insecticidal activity” are used synonymously to refer to activity of an organism or a substance (such as, for example, a protein) that can be measured, by way of non-limiting example, via pest mortality, retardation of pest development, pest weight loss, pest repellency, and other behavioral and physical changes of a pest after feeding and exposure for an appropriate length of time. In this manner, pesticidal activity often impacts at least one measurable parameter of pest fitness. For example, the pesticide or pesticidal property may be a polypeptide to decrease or inhibit insect feeding and/or to increase insect mortality upon ingestion of the polypeptide limit the amount of damage that the pest can cause to a root mass. Assays for assessing pesticidal activity are well known in the art. See, e.g., U.S. Pat. Nos. 6,570,005 and 6,339,144.

As used herein, the term “pesticidal gene” or “pesticidal polynucleotide” refers to a nucleotide sequence that encodes a polypeptide that exhibits pesticidal activity. As used herein, the terms “pesticidal polypeptide,” “pesticidal protein,” or “insect toxin” are intended to mean a protein having pesticidal activity.

As used herein, the term “pesticidally effective amount” connotes a quantity of a substance or organism that has pesticidal activity when present in the environment of a pest. For each substance or organism, the pesticidally effective amount is determined empirically for each pest affected in a specific environment. Similarly an “insecticidally effective amount” may be used to refer to a “pesticidally effective amount” when the pest is an insect pest.

As used herein, the term “plant” includes reference to whole plants, plant organs (e.g., leaves, stems, roots, etc.), seeds, plant cells, plant protoplasts, plant cell tissue cultures from which plants can be regenerated, plant calli, plant clumps, and plant cells that are intact in plants or parts of plants and progeny of same. Parts of transgenic plants are to be understood within the scope of the invention to comprise, for example, plant cells, protoplasts, tissues, callus, embryos as well as flowers, pollen, ovules, seeds, branches, kernels, ears, cobs, husks, stalks, stems, fruits, leaves, roots, root tips, anthers, and the like, originating in transgenic plants or their progeny previously transformed with a DNA molecule of the invention and therefore consisting at least in part of transgenic cells, are also an object of the present invention. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

As used herein, the term “plant cell” includes, without limitation, seeds, suspension cultures, embryos, meristematic regions, callus tissue, leaves, roots, shoots, gametophytes, sporophytes, pollen, and microspores. The class of plants that can be used in the methods of the invention is generally as broad as the class of higher plants amenable to transformation techniques, including both monocotyledonous and dicotyledonous plants.

A “plot” is intended to mean an area where crops are planted, comprising one or more fields or areas of indeterminate size.

As used herein, the term “polypeptide,” “peptide,” and “protein” are used interchangeably herein to refer to a polymer of amino acid residues. The terms apply to amino acid polymers in which one or more amino acid residues is an artificial chemical analogue of a corresponding naturally-occurring amino acid, as well as to naturally-occurring amino acid polymers.

As used herein, “protects a plant from an insect pest” is intended to mean the limiting or eliminating of insect pest-related damage to a plant by, for example, inhibiting the ability of the insect pest to grow, feed, and/or reproduce or by killing the insect pest. As used herein, “impacting an insect pest of a plant” includes, but is not limited to, deterring the insect pest from feeding further on the plant, harming the insect pest by, for example, inhibiting the ability of the insect to grow, feed, and/or reproduce, or killing the insect pest.

A “trait,” as used herein, refers to the phenotype derived from a particular sequence or groups of sequences. A single expression cassette may contain both a nucleotide encoding a pesticidal protein of interest, and at least one additional gene, such as a gene employed to increase or improve a desired quality of the transgenic plant. Alternatively, the additional gene(s) can be provided on multiple expression cassettes. The combinations generated can also include multiple copies of any one of the polynucleotides of interest. Additionally, either a single expression cassette or multiple expression cassettes may encode pesticidal activity or other activities or markers which may be useful in determining trait expression.

As used herein, the term “transgenic” includes any cell, cell line, callus, tissue, plant part, or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, the term “transgenic pest resistant crop plant” means a plant or progeny thereof (including seeds) derived from a transformed plant cell or protoplast, wherein the plant DNA contains an introduced heterologous DNA molecule, not originally present in a native, non-transgenic plant of the same strain, that confers resistance to one or more pests. In a preferred embodiment these pests are rootworms, such as but not limited to, corn rootworms. In another embodiment, these pests are WCRW, NCRW and MCRW. The term refers to the original transformant and progeny of the transformant that include the heterologous DNA. The term also refers to progeny produced by a sexual outcross between the transformant and another variety that includes the heterologous DNA. It is also to be understood that two different transgenic plants can also be mated to produce offspring that contain two or more independently segregating, added, heterologous genes. Selfing of appropriate progeny can produce plants that are homozygous for both added, heterologous genes. Back-crossing to a parental plant and out-crossing with a non-transgenic plant are also contemplated, as is vegetative propagation. Descriptions of other breeding methods that are commonly used for different traits and crop plants can be found in one of several references, e.g., Fehr (1987), in Breeding Methods for Cultivar Development, ed. J. Wilcox (American Society of Agronomy, Madison, Wis.).

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera. Of particular interest are insect pests that damage the roots of maize plants.

Of interest are larvae and adults of the order Coleoptera including cucumber beetles, rootworms, leaf beetles, potato beetles, and leafminers in the family Chrysomelidae (including, but not limited to: Diabrotica virgifera virgifera LeConte (western corn rootworm); D. barberi Smith & Lawrence (northern corn rootworm); D. undecimpunctata howardi Barber (southern corn rootworm); Colaspis brunnea Fabricius (grape colaspis); chafers and other beetles from the family Scarabaeidae (including, but not limited to: Popillia japonica Newman (Japanese beetle); Cyclocephala borealis Arrow (northern masked chafer, white grub); C. immaculata Olivier (southern masked chafer, white grub); Rhizotrogus majalis Razoumowsky (European chafer); Phyllophaga crinita Burmeister (white grub); and wireworms from the family Elateridae, Eleodes spp., Melanotus spp.; Conoderus spp.; Limonius spp.; Agriotes spp.; Ctenicera spp.; Aeolus spp.

Pests can also include other invertebrates, such as nematodes, and plant diseases of plant roots, such as, for example, diseases of bacterial, fungal, or other origin that affect root health.

### Sequential Sampling Advantages

Exemplary embodiments utilize an improved method for determining resistance of a pest to pesticidal activity of transgenic pest resistant crop plants in a plot. This improvement is provided through sequential sampling and involves choosing plants, determining trait expression in the plants, and rating each of the plants that is determined to express the trait for damage caused by the pest.

In sequential sampling, sample size is not predetermined, but rather choosing of plants occurs, the chosen plants are tested for trait expression, and plants testing positive for pesticidal activity via trait expression make up a batch. The batch is evaluated for root damage and, based upon the ratings occurring after each batch, another batch may be needed in order to determine pest resistance. This method is an improvement on standard methods as fewer numbers of plants are required for determining resistance by sampling in smaller numbers (batches), evaluating data after each batch. For example, the traditional sampling recommendation for a 100 acre plot would dictate that 14-20 samples be collected (Integrated Pest Management; Intro to Crop Scouting, Missouri University extension office, Extension Publication Plant Protection Programs, 2800 Maguire Blvd, Columbia, Mo. 65211) while with sequential sampling would utilize as few as 5 plants per 100 acres. Additional plants can also be sampled, such that the number of plants sampled may be as many as 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 plants.

### Choosing a Plant

Choosing plants for sampling for integrated pest monitoring (IPM) may be performed using an unbiased but not completely randomized scheme. This keeps the majority of the sampling from being located in the same area of a field by imposing restrictions on the sampling pattern. These restrictions help ensure that all areas of the field are sampled in an unbiased manner. Furthermore, sampling schemes are varied and may have one or more pest, host and field influences. In addition, the sampling pattern is also influenced to be centrally implemented to be representative of the area inside of the field border as defined by the sampling protocol. Also, the samples are preferably taken at a minimum distance from each other and in some cases no two samples should be taken within a set distance from each other as defined by the sampling protocol. A map of the field is especially helpful when deciding where to sample. As a predetermined sampling point is reached, a plant a set number of rows or plants from the sampling point is chosen for sampling. If any plants adjacent to it are volunteer, refuge plants, or are determined to be negative in the appropriate trait expression assay, then an alternate sampling point is chosen that is a set distance away. Another plant is then chosen, using the same criteria.

It is recognized that a variety of different methods can be used to provide an unbiased sampling of plants. It is further recognized that certain methods can be appropriate for a particular plot having, for example, a particular plot area and shape but that such methods might not be appropriate for a plot of different size and shape. One of skill in the art will be able to select and use in the methods disclosed herein, one or more methods for the sampling of plants that is appropriate for any particular plot and is unbiased.

Methods of sampling include, but are not limited to, zig-zag, M-pattern, and circles, however any unbiased sampling method may be used. In one embodiment, sampling is based on the M pattern, a common pattern in scouting programs involving walking along a predetermined M-shaped route through a field. See I.P.M.; Intro to Crop Scouting, M.U. Extension Office). In this embodiment, a representative number of plants chosen for sampling in an unbiased sampling manner, comprises 5-19 plants per 100 acres, further comprises 5-16 plants per 100 acres, is preferably 5-14 plants per 100 acres, is more preferably 5-10 plants per 100 acres and is most preferably 5 plants per 100 acres. In another embodiment, sampling is based on a circle; a common pattern in plot over 150 acres involving walking in a circle-based method through a field.

Taking smaller samples saves time, money, plants, materials, and potential damage to the plot. Thus, the methods of the present invention provide an improvement over existing methods of sampling plants for determining the resistance of a pesticidal activity of a pest resistant crop plant, particularly a transgenic pest resistant crop plant.

### Determining Trait Expression

After plants are chosen, trait expression is determined in order to identify plants having pesticidal activity which then are considered a batch.

Determining trait expression in the transgenic pest resistant plants uses methods known to one of skill in the art. Any assay, or combination, appropriate for trait expression may be used. Assays may detect protein, RNA, reaction products, or utilize markers, be visual, require instrumentation or not. These assays may include any useful means of detecting trait expression or combination thereof. In one embodiment the assay may be an immunoassay, histochemical stain, or enzymatic process or product. In a preferred embodiment, the assay is an ELISA such as, for example, an ELISA strip test.

Resistance to root pests can be introduced into the crop plant by any method known in the art. In some embodiments, the different modes of pesticidal activity include toxin binding to different binding sites in the gut membranes of the corn rootworms. Transgenes in the present invention, useful against rootworms include, but are not limited to, those encoding Bt proteins. Other transgenes and stacked genes appropriate for other pests are also discussed herein and are known to one skilled in the art. A non-limiting example of such a gene is a gene that encodes a Bt toxin, such as a homologue of a known Cry toxin. “Bt toxin” is intended to mean the broader class of toxins found in various strains of Bt, which includes such toxins as, for example, the vegetative insecticidal proteins and the δ-endotoxins. See, e.g., Crickmore et al. (1998) Microbiol. Molec. Biol. Rev. 62:807-813. Stacked genes in plants of the embodiments may also encode polypeptides having insecticidal activity other than Bt toxic proteins, such as lectins (Van Damme et al. (1994) Plant Mol. Biol. 24:825, pentin (described in U.S. Pat. No. 5,981,722), lipases (lipid acyl hydrolases, see, e.g., those disclosed in U.S. Pat. Nos. 6,657,046 and 5,743,477; see also WO2006131750A2), cholesterol oxidases from Streptomyces, and pesticidal proteins derived from Xenorhabdus and Photorhabdus bacteria species, Bacillus laterosporus species, and Bacillus sphaericus species, and the like. Also contemplated is the use of chimeric (hybrid) toxins (see, e.g., Bosch et al. (1994) Bio/Technology 12:915-918).

Such transformants can contain transgenes that are derived from the same class of toxin (e.g., more than one δ-endotoxin, more than one pesticidal lipase, more than one binary toxin, and the like), or the transgenes can be derived from different classes of toxins (e.g., a δ-endotoxin in combination with a pesticidal lipase or a binary toxin). For example, a plant having the ability to express an insecticidal δ-endotoxin derived from Bt (such as Cry1F), also has the ability to express a pesticidal lipase, such as, for example, a lipid acyl hydrolase.

In practice, certain stacked combinations of the various Bt and other genes described previously are best suited for certain pests, based on the nature of the pesticidal action and the susceptibility of certain pests to certain toxins. For example, some transgenic combinations are particularly suited for use against various types of CRW, including WCRW, NCRW, and MCRW. These combinations include at least Cry34/35 and Cry3A; and Cry34/35 and Cry3B. Other combinations are also known for other pests. Also, various combinations may be further combined with each other in order to provide resistance management to multiple pests.

In one embodiment trait expression is determined for the plants chosen through the unbiased sampling manner. Transgenic pest resistant crop plants are assayed in or near the plot, but prior to rating roots for pest damage. In this preferred embodiment, a pest damage rating of roots is performed on plants having confirmed pesticidal activity. In another embodiment, assays may be preliminary assays and results may be, but aren't limited to, supplementation and further testing.

In one embodiment, assay methods are adapted for field testing so that plants for the batch may be identified and analyzed for pest damage. In another embodiment, assay methods consist of special field equipment. In yet another embodiment, assays are done via remote, long distance or transmission equipment. Assays for trait activity may be performed on any part of the plant, such as but not limited to root, leaf, tassel, silk, shoot or a combination thereof. In a preferred embodiment, assays may be performed on tissue such as but not limited to green leaf or root tissue.

In one embodiment, the trait expression detected is a pesticidal activity. This would identify plants having pesticidal activity such as, but not limited to that associated with expression of a Bt gene such as Cry34/35, other Cry genes, genes encoding vegetative insecticidal proteins (VIP), and combinations thereof. A further embodiment provides these or other pesticidal genes stacked with a Bt gene. In yet another embodiment, trait expression methods detect two or more different modes of pesticidal action resulting from planting a seed mixture. If assay results are negative for an individual plant, unbiased sampling methods are used and another plant is assayed for trait expression.

In another embodiment, the trait expression is not limited to pesticidal activity. Trait expression assays could detect, for example, a co-expressed, hybrid or co-activity directly linked to pesticidal activity. Detection may also be of a marker gene or a co-expressed gene that is indirectly linked to pesticidal activity. For example, if trait expression assays are performed on plants transformed with a bar gene, the transgenic plants can be painted with bialaphos herbicide (1% v/v Liberty). Also trait expression can be detected via ELISA, PCR, Green Fluorescent Protein or a combination thereof. Other ways to measure trait detection are known to those of skill in the art.

In yet another embodiment, trait expression may reflect more than one pesticidal activity, and other trait expression, or a combination thereof.

Preferably, the number of plants chosen will be equal to the number of plants in the batch. However, if one or more plants lack pesticidal activity, then the number of plants chosen will be greater than the number in the batch. In another embodiment, if one or more plants lack trait expression, then the number of plants chosen will also be greater than the number in the batch. In one embodiment a batch comprises 50, 40, 30, 20, 15, 13, 12, 11, 10, 9, 8, 7, 6 or 5 plants. In another embodiment, a batch comprises plants in fewer numbers than taken in traditional non-unbiased sampling methods. For example, the number of plants in a batch may comprise at least 5 but fewer than 20 plants per 100 acres, at least 5 but fewer than 15 plants per 100 acres, at least 5 but fewer than 10 plants per 100 acres, at least 5 but fewer than 8 plants per 100 acres, or 5 plants per 100 acres. In one embodiment, sequential sampling utilizes smaller than usual, but statistically significant, sample sizes. In another embodiment, the sequential sampling size is a batch which is 5 or fewer plants per 100 acres. Some embodiments also provide methods of sampling utilizing 10 or fewer samples per 100 acres of a plot.

Sequential sampling can be used in lieu of (or in addition to) data collected using more plants to determine resistance of a pest with sufficient confidence to determine if pest resistance is suggested or not. In one embodiment, a batch of 5 plants per 100 acres is sufficient to determine pest resistance. In another embodiment, a second batch of 5 plants per 100 acres is sufficient to determine pest resistance. In another embodiment, the number of plants needed to determine resistance is smaller in sequential sampling than in traditional sampling methods.

### Rating

Once plants are chosen and have been determined to have pesticidal activity, they are considered a batch and are ready to be rated. Each plant in the batch then has a root rating assigned based upon a modified condition of the root nodes as the indicator of pest damage. For CRW damage, each plant in a batch is rated using a node-injury scale (NIS) (J. D. Oleson, Y. Park, T. M. Nowatzki, and J. J. Tollefson. 2005. J. Econ. Entomol. 98:1-8).

A node of roots is a ring of roots originating from the main stalk and pest damage is assessed by looking at damage to the node. A nodal root is considered damaged, or pruned, if it is eaten back to within approximately 1.5 inches from the stalk or soil line. The node injury scale (NIS, Oleson et al. 2005) ranges from zero to three nodes reflecting the number of roots of each of three nodes damaged. Wherein a zero score indicates the “perfect root mass” with no scarring or feeding damage, a score of 1.0 means that one node has all roots damaged to within 1.5 inches, a score of 2.0 means that two nodes are so damaged and a score of 3.0 is the highest level and indicates that all 3 nodes are so damaged. If the damage is between these whole number levels, this is noted as the “percentage” of the node that is injured, i.e., 1.50 means one and a half nodes are so damaged; 0.25 means one quarter of a node is so damaged.

The NIS for CRW also does not consider aerial brace roots as nodal roots if they have not penetrated the soil, even if there is damage. In general, if any root is damaged by rootworm it most likely will also have brown necrotic tissue, while a root damaged mechanically will not. Other symptoms of rootworm feeding are scarring, tunneling and channeling. Sometimes root regrowth, a secondary root growth typical after peak larval feeding, can hide rootworm damage especially in late-season (after R1) evaluations. Actual cutting away of regrowth may be needed to see rootworm damage. Also, low levels of feeding scars and/or minor root pruning are possible, especially when pest pressure is low. A high level of pruning is expected to be, but also is not necessarily, indicative of the presence of resistant pests.

In order to rate the pest damage to the roots, a plant having pesticidal activity is cut off 1-2 feet from the ground and the whole root mass is dug out with soil shaken off to reveal as much of the root structure as possible. In a preferred embodiment roots are rated in the field without washing. Each of the plants of the batch is dug out and rated for root pest damage. Preferably, this happens at each of the 5 points of the “M” pattern. In another embodiment roots are rated after washing soil off to make them more visible and more easily rated. If this washing happens out of the field, then each plant location and root rating is recorded according to the sampling pattern. In either case, roots missing at or above the predetermined limit score are used to evaluate pest resistance in the plot. In one mode data is used to calculate a composite score for the plot.

In this CRW example, when analyzing the data, if 3-5 of 5 scored roots have node-injury scores of greater than or equal to 0.5 then resistant pests are suspected to be present in the plot. If 0 of 5 scored roots have node-injury scores of greater than or equal to 0.5 then the plot is considered negative for resistant pests. Node-injury scores are inconclusive when 1-2 of 5 scored roots are greater than or equal to 0.5 and another batch of samples should be taken to determine resistance of the pest.

When the sample results of the first 5 plants are inconclusive, the same method for sequential sampling is repeated wherein a second batch of plants is chosen, analyzed for trait expression, and rated for pest damage. If there are 5 or more samples, among the now 10, that have NIS of greater than or equal to 0.5, then there is possible pest resistance. If there are fewer than 2 plants at that level then there is no indication of unexpected damage in the plot. At 2-4 plants, results are inconclusive and further examination of the plot is necessary and another 5 plants should be examined. If results are still inconclusive after looking at a total of twenty plants, then sampling can stop and the field should be marked for future follow up in the next growing season.

Exemplary embodiments utilize methods for determining resistance of a pest to a pesticidal activity so as to avoid development of resistance in the pest to, for example, pesticidal plants expressing Bt toxins. In other embodiments these methods are employed when unexpected damage is detected in a plot, to extend usefulness, extend trait longevity and to delay monogenic resistance evolution. In all embodiments, the predetermined set limit is based on efficacy data generated for the pesticidal transgenic trait.

### Examples

Embodiments of this invention can be better understood by reference to the following examples. The foregoing and following description of embodiments of the present invention and the various embodiments are not intended to limit the claims, but rather are illustrative thereof. Therefore, it will be understood that the claims are not limited to the specific details of these examples. It will be appreciated by those skilled in the art that other embodiments of the invention may be practiced without departing from the spirit and the scope of the disclosure, as defined by the appended claims. The disclosure of each reference set forth herein is incorporated herein by reference in its entirety for all purposes.

### Example 1

### Spatial Correlations and Sampling Distance

CRW Node Injury Score (NIS) data are spatially correlated. A total of 16 research locations from 2005 to 2007 at which the average check score was above 1.0 were surveyed. The plot-to-plot NIS correlations among rows (ranges) ranged from 0.11 to 0.92, with an average of 0.51. The plot-to-plot NIS correlations among columns (plots) ranged from 0.41 to 0.92, with an average of 0.72. See, Table 1.

Since the spatial correlations in both the row direction and the column direction can be as high as 0.9×, in order to make sure the sampled plants are not subjected to the high positive spatial correlation, it is not recommended to sample plants from a neighboring area. For the studied small research plots, the plot size is four 30-inch rows by 17′5-feet row length. That is, the distance from the center of a plot to the center of its neighboring plot on the same row is 30 inches/row×4 rows=120 inches (=10 feet), and the distance from the center of a plot to the center of its neighboring plot on the same column is 17′5 feet. Assume the correlation between two neighboring plots on the same row is 0.95, then the correlation between two plots two columns apart on the same row is 0.95², and the correlation between two plots n columns apart on the same row is 0.95ⁿ. When n=24, the correlation is 0.95²⁴<0.30. Therefore, to get away from the spatial correlation, one must take two samples at least 24 columns apart, which is a distance of at least 10 feet/column×24 columns=240 feet. Again assume the correlation between two neighboring plots on the same column is 0.95, and then the correlation is lowered to <0.30 when two plots are at least 24 ranges apart on the same column, which equals to a distance of at least 17′5 feet/range×24 ranges=418 feet.

The safety distances of 418-feet long and 240-feet wide were derived from the small research plots. To be absolutely safe from the spatial correlation in large plots (strip plots), one may take a sampling point as the center of a circle, and move at least 500 feet in any direction to take another sample. Such circles can serve as sampling units. The acreage of such a circle is about 18 acres (π×500²=785,398 feet², 1 acre=43,560 feet²). Therefore, for fields larger than 150 acres, samples are taken of plants across the entire field for each 100 acres meets the safety distance requirement of at least 18 acres.

TABLE 1
Spatial Correlations and Sampling Distance

| | Location | Field | | | | | Non-Reliable |
|---|---|---|---|---|---|---|---|
| Year | Name | Size | CovParm | Group | Interpretation | Estimate | Estimate |
| 2005 | JHTBCC | 18 × 17 | Variance | | plot-to-plot variation | 0.007 | |
| 2005 | JHTBCC | 18 | SP(POWA) RNG | | spatial correlation between neighboring ranges | 0.144 | |
| 2005 | JHTBCC | 17 | SP(POWA) PLT | | spatial correlation between neighboring rows | 0.490 | |
| 2005 | JHTBCC | | Residual | trt_seg Check | within plot variation among sampled plants | 0.411 | |
| 2005 | JHTBCC | | Residual | trt_seg HXRW | within plot variation among sampled plants | 0.070 | |
| 2005 | JHTBCC | | Residual | trt_seg HXX | within plot variation among sampled plants | 0.131 | |
| 2005 | JHTBCC | | Residual | trt_seg YGPL | within plot variation among sampled plants | 0.080 | |
| 2005 | JHTBCC | | Residual | trt_seg YGRW | within plot variation among sampled plants | 0.032 | |
| 2005 | JVTM01 | 3 × 51 | Variance | | plot-to-plot variation | 0.063 | |
| 2005 | JVTM01 | 3 | SP(POWA) RNG | | spatial correlation between neighboring ranges | 0.368 | |
| 2005 | JVTM01 | 51 | SP(POWA) PLT | | spatial correlation between neighboring rows | −0.004 | X |
| 2005 | JVTM01 | | Residual | trt_seg Check | within plot variation among sampled plants | 0.084 | |
| 2005 | JVTM01 | | Residual | trt_seg HXRW | within plot variation among sampled plants | 0.188 | |
| 2005 | JVTM01 | | Residual | trt_seg HXX | within plot variation among sampled plants | 0.192 | |
| 2005 | JVTM01 | | Residual | trt_seg YGPL | within plot variation among sampled plants | 0.067 | |
| 2005 | JVTM01 | | Residual | trt_seg YGRW | within plot variation among sampled plants | 0.075 | |
| 2005 | MKTB3F | 13 × 8 | Variance | | plot-to-plot variation | 0.000 | |
| 2005 | MKTB3F | 13 | SP(POWA) RNG | | spatial correlation between neighboring ranges | 0.868 | X |
| 2005 | MKTB3F | 8 | SP(POWA) PLT | | spatial correlation between neighboring rows | 0.846 | X |
| 2005 | MKTB3F | | Residual | trt_seg Check | within plot variation among sampled plants | 0.353 | |
| 2005 | MKTB3F | | Residual | trt_seg HXRW | within plot variation among sampled plants | 0.007 | |
| 2005 | MKTB3F | | Residual | trt_seg HXX | within plot variation among sampled plants | 0.005 | |
| 2005 | MKTB3F | | Residual | trt_seg YGPL | within plot variation among sampled plants | 0.016 | |
| 2005 | MKTB3F | | Residual | trt_seg YGRW | within plot variation among sampled plants | 0.044 | |
| 2005 | MRT37A | 4 × 30 | Variance | | plot-to-plot variation | 0.011 | |
| 2005 | MRT37A | 4 | SP(POWA) RNG | | spatial correlation between neighboring ranges | 0.307 | |
| 2005 | MRT37A | 30 | SP(POWA) PLT | | spatial correlation between neighboring rows | −0.203 | X |
| 2005 | MRT37A | | Residual | trt_seg Check | within plot variation among sampled plants | 0.102 | |
| 2005 | MRT37A | | Residual | trt_seg HXRW | within plot variation among sampled plants | 0.079 | |
| 2005 | MRT37A | | Residual | trt_seg HXX | within plot variation among sampled plants | 0.115 | |
| 2005 | MRT37A | | Residual | trt_seg YGPL | within plot variation among sampled plants | 0.024 | |
| 2005 | MRT37A | | Residual | trt_seg YGRW | within plot variation among sampled plants | 0.006 | |
| 2005 | RHTE11 | 4 × 37 | Variance | | plot-to-plot variation | 0.001 | |
| 2005 | RHTE11 | 4 | SP(POWA) RNG | | spatial correlation between neighboring ranges | 1.000 | X |
| 2005 | RHTE11 | 37 | SP(POWA) PLT | | spatial correlation between neighboring rows | 0.569 | X |
| 2005 | RHTE11 | | Residual | trt_seg Check | within plot variation among sampled plants | 0.808 | |
| 2005 | RHTE11 | | Residual | trt_seg HXRW | within plot variation among sampled plants | 0.141 | |
| 2005 | RHTE11 | | Residual | trt_seg HXX | within plot variation among sampled plants | 0.244 | |
| 2005 | RHTE11 | | Residual | trt_seg YGPL | within plot variation among sampled plants | 0.074 | |
| 2005 | RHTE11 | | Residual | trt_seg YGRW | within plot variation among sampled plants | 0.003 | |
| 2005 | WNT15B | 11 × 14 | Variance | | plot-to-plot variation | 0.000 | X |
| 2005 | WNT15B | 11 | SP(POWA) RNG | | spatial correlation between neighboring ranges | 0.726 | X |
| 2005 | WNT15B | 14 | SP(POWA) PLT | | spatial correlation between neighboring rows | 0.782 | X |
| 2005 | WNT15B | | Residual | trt_seg Check | within plot variation among sampled plants | 0.345 | |
| 2005 | WNT15B | | Residual | trt_seg HXRW | within plot variation among sampled plants | 0.007 | |
| 2005 | WNT15B | | Residual | trt_seg HXX | within plot variation among sampled plants | 0.002 | |
| 2005 | WNT15B | | Residual | trt_seg YGPL | within plot variation among sampled plants | 0.027 | |
| 2005 | WNT15B | | Residual | trt_seg YGRW | within plot variation among sampled plants | 0.047 | |
| 2005 | YKTB02 | 3 × 50 | Variance | | plot-to-plot variation | 0.001 | X |
| 2005 | YKTB02 | 3 | SP(POWA) RNG | | spatial correlation between neighboring ranges | 0.610 | X |
| 2005 | YKTB02 | 50 | SP(POWA) PLT | | spatial correlation between neighboring rows | 0.740 | X |
| 2005 | YKTB02 | | Residual | trt_seg Check | within plot variation among sampled plants | 0.651 | |
| 2005 | YKTB02 | | Residual | trt_seg HXRW | within plot variation among sampled plants | 0.007 | |
| 2005 | YKTB02 | | Residual | trt_seg HXX | within plot variation among sampled plants | 0.004 | |
| 2005 | YKTB02 | | Residual | trt_seg YGPL | within plot variation among sampled plants | 0.029 | |
| 2005 | YKTB02 | | Residual | trt_seg YGRW | within plot variation among sampled plants | 0.028 | |
| 2005 | WNT14B | | Residual | trt_seg Check | within plot variation among sampled plants | 0.293 | |
| 2005 | WNT14B | | Residual | trt_seg HXRW | within plot variation among sampled plants | 0.004 | |
| 2005 | WNT14B | | Residual | trt_seg HXX | within plot variation among sampled plants | 0.004 | |
| 2005 | WNT14B | | Residual | trt_seg YGPL | within plot variation among sampled plants | 0.062 | |
| 2005 | WNT14B | | Residual | trt_seg YGRW | within plot variation among sampled plants | 0.050 | |
| 2006 | AGBNBA | 7 × 75 | Variance | | plot-to-plot variation | 0.003 | |
| 2006 | AGBNBA | 7 | SP(POWA) RNG | | spatial correlation between neighboring ranges | 0.777 | |
| 2006 | AGBNBA | 75 | SP(POWA) PLT | | spatial correlation between neighboring rows | 0.915 | |
| 2006 | AGBNBA | | Residual | trt_seg Check | within plot variation among sampled plants | 0.429 | |
| 2006 | AGBNBA | | Residual | trt_seg HXRW | within plot variation among sampled plants | 0.017 | |
| 2006 | AGBNBA | | Residual | trt_seg HXX | within plot variation among sampled plants | 0.013 | |
| 2006 | AGBNBA | | Residual | trt_seg YGPL | within plot variation among sampled plants | 0.096 | |
| 2006 | AGBNBA | | Residual | trt_seg YGRW | within plot variation among sampled plants | 0.139 | |
| 2006 | MKBN05 | 9 × 58 | Variance | | plot-to-plot variation | 0.018 | |
| 2006 | MKBN05 | 9 | SP(POWA) RNG | | spatial correlation between neighboring ranges | 0.113 | |
| 2006 | MKBN05 | 58 | SP(POWA) PLT | | spatial correlation between neighboring rows | 0.790 | |
| 2006 | MKBN05 | | Residual | trt_seg Check | within plot variation among sampled plants | 0.240 | |
| 2006 | MKBN05 | | Residual | trt_seg HXRW | within plot variation among sampled plants | 0.058 | |
| 2006 | MKBN05 | | Residual | trt_seg HXX | within plot variation among sampled plants | 0.060 | |
| 2006 | MKBN05 | | Residual | trt_seg YGPL | within plot variation among sampled plants | 0.050 | |
| 2006 | MKBN05 | | Residual | trt_seg YGRW | within plot variation among sampled plants | 0.061 | |
| 2006 | RHBLCR | 14 × 47 | Variance | | plot-to-plot variation | 0.009 | |
| 2006 | RHBLCR | 14 | SP(POWA) RNG | | spatial correlation between neighboring ranges | 0.909 | |
| 2006 | RHBLCR | 47 | SP(POWA) PLT | | spatial correlation between neighboring rows | 0.763 | |
| 2006 | RHBLCR | | Residual | trt_seg Check | within plot variation among sampled plants | 0.776 | |
| 2006 | RHBLCR | | Residual | trt_seg HX1 | within plot variation among sampled plants | 0.679 | |
| 2006 | RHBLCR | | Residual | trt_seg HXRW | within plot variation among sampled plants | 0.020 | |
| 2006 | RHBLCR | | Residual | trt_seg HXX | within plot variation among sampled plants | 0.030 | |
| 2006 | RHBLCR | | Residual | trt_seg YGPL | within plot variation among sampled plants | 0.068 | |
| 2006 | RHBLCR | | Residual | trt_seg YGRW | within plot variation among sampled plants | 0.002 | |
| 2006 | YKBF04 | 59 × 12 | Variance | | plot-to-plot variation | 0.023 | |
| 2006 | YKBF04 | 59 | SP(POWA) RNG | | spatial correlation between neighboring ranges | 0.924 | |
| 2006 | YKBF04 | 12 | SP(POWA) PLT | | spatial correlation between neighboring rows | 0.856 | |
| 2006 | YKBF04 | | Residual | trt_seg Check | within plot variation among sampled plants | 0.327 | |
| 2006 | YKBF04 | | Residual | trt_seg HXRW | within plot variation among sampled plants | 0.032 | |
| 2006 | YKBF04 | | Residual | trt_seg HXX | within plot variation among sampled plants | 0.052 | |
| 2006 | YKBF04 | | Residual | trt_seg YGPL | within plot variation among sampled plants | 0.085 | |
| 2006 | YKBF04 | | Residual | trt_seg YGRW | within plot variation among sampled plants | 0.026 | |
| 2006 | ENBLRW | | Residual | trt_seg Check | within plot variation among sampled plants | 0.344 | |
| 2006 | ENBLRW | | Residual | trt_seg HX1 | within plot variation among sampled plants | 0.374 | |
| 2006 | ENBLRW | | Residual | trt_seg HXRW | within plot variation among sampled plants | 0.017 | |
| 2006 | ENBLRW | | Residual | trt_seg HXX | within plot variation among sampled plants | 0.026 | |
| 2006 | ENBLRW | | Residual | trt_seg YGPL | within plot variation among sampled plants | 0.016 | |
| 2006 | ENBLRW | | Residual | trt_seg YGRW | within plot variation among sampled plants | 0.033 | |
| 2006 | MRBNRE | | Residual | trt_seg Check | within plot variation among sampled plants | 0.393 | |
| 2006 | MRBNRE | | Residual | trt_seg HX1 | within plot variation among sampled plants | 0.480 | |
| 2006 | MRBNRE | | Residual | trt_seg HXRW | within plot variation among sampled plants | 0.028 | |
| 2006 | MRBNRE | | Residual | trt_seg HXX | within plot variation among sampled plants | 0.049 | |
| 2006 | MRBNRE | | Residual | trt_seg YGPL | within plot variation among sampled plants | 0.052 | |
| 2006 | MRBNRE | | Residual | trt_seg YGRW | within plot variation among sampled plants | 0.135 | |
| 2007 | CIYNBC | 9 × 4 | Variance | | plot-to-plot variation | 0.028 | |
| 2007 | CIYNBC | 9 | SP(POWA) RNG | | spatial correlation between neighboring ranges | 0.517 | X |
| 2007 | CIYNBC | 4 | SP(POWA) PLT | | spatial correlation between neighboring rows | 0.834 | |
| 2007 | CIYNBC | | Residual | trt_seg Check | within plot variation among sampled plants | 0.588 | |
| 2007 | CIYNBC | | Residual | trt_seg HXX | within plot variation among sampled plants | 0.031 | |
| 2007 | CIYNBC | | Residual | trt_seg YGPL | within plot variation among sampled plants | 0.019 | |
| 2007 | RHYLCR | 12 × 3 | Variance | | plot-to-plot variation | 0.074 | |
| 2007 | RHYLCR | 12 | SP(POWA) RNG | | spatial correlation between neighboring ranges | 0.847 | X |
| 2007 | RHYLCR | 3 | SP(POWA) PLT | | spatial correlation between neighboring rows | 0.409 | |
| 2007 | RHYLCR | | Residual | trt_seg Check | within plot variation among sampled plants | 0.701 | |
| 2007 | RHYLCR | | Residual | trt_seg HXX | within plot variation among sampled plants | 0.057 | |
| 2007 | RHYLCR | | Residual | trt_seg YGPL | within plot variation among sampled plants | 0.240 | |

### Example 2

### Sequential Sampling Plan

To develop a sequential sampling plan, it is necessary to find the lower and upper thresholds (as percentages), p₁, p₂. They represent the lower and upper limits for percentages of samples with NIS scores at or beyond 0.5. A total of 153 hybrids with CRW protection (i.e. containing any one of the following trait segments: HXRW, HXX, YGPL, YGRW) were surveyed at a total of 16 locations from 2005 to 2007. The Least Square Mean (LSMean) of NIS score, together with the lower and upper confidence limits for the 95% confidence interval of LSMean, for each hybrid across locations were output from SAS proc mixed. If the upper confidence limit is above 0.5, and the lower confidence limit is above 0, the hybrid is deemed to be a problematic hybrid in terms of its resistance to the CRW. Ten such hybrids were found. See, Table 2.

TABLE 2
Percentages and Means from Historical Research Data

| | | CRWNIS | | CRWNIS | CRWNIS | n | nCRWINS ≧ | nCRWINS ≧ |
|---|---|---|---|---|---|---|---|---|
| Hybrid | Trait | LSMean | StdErr | LCL95 | UCL95 | total | 0.5% | 0.5 |
| 1 | A | 0.30 | 0.13 | 0.04 | 0.55 | 33 | 6 | 18 |
| 2 | B | 0.40 | 0.08 | 0.23 | 0.56 | 30 | 7 | 23 |
| 3 | C | 0.46 | 0.08 | 0.30 | 0.61 | 25 | 7 | 28 |
| 4 | D | 0.35 | 0.11 | 0.13 | 0.67 | 31 | 9 | 29 |
| 5 | C | 0.42 | 0.07 | 0.28 | 0.56 | 51 | 15 | 29 |
| 6 | A | 0.33 | 0.13 | 0.07 | 0.60 | 30 | 9 | 30 |
| 7 | D | 0.31 | 0.11 | 0.09 | 0.54 | 30 | 10 | 33 |
| 8 | B | 0.34 | 0.08 | 0.17 | 0.51 | 29 | 10 | 34 |
| 9 | B | 0.49 | 0.08 | 0.33 | 0.65 | 35 | 18 | 51 |
| 10 | A | 0.89 | 0.07 | 0.76 | 1.02 | 49 | 28 | 57 |

Among the 10 hybrids, the lowest percentage of samples with NIS score ≧0.5 was 18%, which was found for hybrid 1. A total of 33 plants were taken from 3 locations in 2005, and 6 of them had a NIS score greater or equal to 0.5. The confidence intervals for NIS mean score was (0.04, 0.55). Since the precision of percentage out of 33 was 3% (i.e. = 1/33×100%), we decided to set the lower threshold p₁=20%. Among the 10 hybrids, the last 2 hybrids' NIS means approached or exceeded 0.5. To be more conservative, we took the statistics for hybrid 9 as the upper limit. The percentage of samples with NIS score ≧0.5 was 51%. A total of 35 plants were taken from 4 locations in 2006, and 18 of them had a NIS score greater or equal to 0.5. Since the precision of percentage out of 35 was 3%, we decided to set the upper threshold p₂=50%.

As for a classical fixed sampling plan, it is also necessary to pre-define the two probabilities of error for a sequential sampling plan. To be more conservative to the customers, we suggest to maintain a low type II error (false-negative error) and high power, 1−β>=0.90. To start with 5 samples, we set the type I error (false-positive error) α=0.20 and 1−β=0.90, and as we increase sampling, we reduced the type I error gradually to α=0.05, and increased the power to 1−β=0.95. Table 3 below shows the suggested sampling plan:

TABLE 3
Sequential Sampling Plan for Field Implementation

| n | alpha | power | high resistance | low resistance | no conclusion |
|---|---|---|---|---|---|
| 5 | 0.20 | 0.90 | 0 | ≧3 | 1-2: continue |
| 10 | 0.15 | 0.95 | ≦1 | ≧5 | 2-4: continue |
| 15 | 0.10 | 0.95 | ≦3 | ≧7 | 4-6: continue |
| 20 | 0.05 | 0.95 | ≦4 | ≧9 | 5-8: stop |

For a given hybrid, 5 random samples (i.e. 5 plants) are taken from the field, with each two samples at least 18 acres apart. Among the 5 samples, if none scores greater or equal to 0.5, this hybrid can be deemed as high resistant to CRW and does not have any problem. If among the 5 samples, there are 3 or more samples with NIS scores greater or equal to 0.5, this hybrid is deemed as low resistant to CRW and the problem is indicated. If among the 5 samples, there are 1 or 2 samples with NIS scores greater or equal to 0.5, no conclusion can be made at this point, and the sampling procedure should be repeated. Among the accumulated 10 samples, if only 1 sample has a score greater or equal to 0.5, this hybrid can be deemed as having no problem. If 5 or more samples have a score greater or equal to 0.5, a problem is indicated. If 2 to 4 samples have a score greater or equal to 0.5, no conclusion can be made and the sampling procedure should be repeated. The maximal number of samples taken is suggested to be 20. If 20 samples are taken and still no conclusion can be made, note this field/farm location for follow up monitoring this hybrid the following year.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method of sequential sampling for determining resistance of a pest to a pesticidal activity of a transgenic pest resistant crop plant in a plot comprising the steps of:
(a) choosing at least about 5 plants in a plot in an unbiased sampling manner appropriate for said plot; wherein said plot comprises transgenic pest resistant crop plants;
(b) determining trait expression in said plants to identify plants having pesticidal activity to at least one target pest to produce a batch comprising at least about 5 and no more than about 50 plants per 100 acres; and
(c) rating the pest damage to a plurality of root masses of said batch; and
(d) evaluating said rating score to determine the resistance of said target pest in said plot.

2. The method of claim 1, wherein said crop plant is maize.

3. The method of claim 1, wherein said target pest is a root pest.

4. The method of claim 1, wherein said pesticidal activity is derived from a protein selected from the group consisting of Cry1A, Cry1A(a), Cry1A(b), Cry1A(c), Cry1C, Cry1D, Cry1E, Cry1F, Cry2A, Cry3Bb, Cry3Bb1, Cry5, Cry8, Cry9C, Cry34, Cry35, as well as functional fragments, functional chimeric, functional shuffled modifications, other functional variants or a combination thereof.

5. The method of claim 1, wherein said pest damage is to a said root mass.

6. The method of claim 3, wherein said root pest is selected from the group consisting of: western corn rootworm, northern corn rootworm, Mexican corn rootworm, scarab beetle larvae, wireworm larvae, nematodes, or a combination thereof.

7. The method of claim 3, wherein the root pest is a plant disease, wherein such plant disease affects root health.

8. The method of claim 1, wherein said batch comprises at least about 5 plants but fewer than about 20 plants per 100 acres.

9. The method of claim 1, wherein said batch comprises at least about 5 plants but fewer than about 15 plants per 100 acres.

10. The method of claim 1, wherein said batch comprises at least about 5 plants but fewer than about 10 plants per 100 acres.

11. The method of claim 1, wherein said batch comprises at least about 5 plants but fewer than about 8 plants per 100 acres.

12. The method of claim 1, wherein said batch comprises 5, 6, 7, 8, 9, or 10 plants.

13. The method of claim 1, wherein said trait expression detection includes protein, RNA, reaction products, or combinations thereof.

14. The method of claim 1, wherein said trait expression is a pesticidal activity.

15. The method of claim 1, wherein said pesticidal activity is detected by ELISA.

16. The method of claim 1, wherein said trait expression is detected using a marker.

17. The method of claim 1, further comprising repeating the steps of claim 1 one time per 100 acres.

18. The method of claim 1, further comprising repeating the steps of claim 1 two or more times per 100 acres.

19. A method of sequential sampling for determining resistance of a pest to a pesticidal activity of a transgenic pest resistant crop plant in a plot comprising the steps of:
(a) choosing at least about 5 plants in a plot in an unbiased sampling manner appropriate for said plot; wherein said plot comprises transgenic pest resistant crop plants;
(b) determining trait expression in said plants to identify plants having pesticidal activity to produce a batch at least about 5 plants; and
(c) rating the pest damage to a root of said batch to determine a composite score and wherein said composite score is greater than or equal to 2 or greater than or equal to 4, after repeating the steps of choosing, determining and rating, then pests may be resistant to said pesticidal activity.

20. A method of sampling a field of maize to determine root damage comprising:
(a) selecting at least five plants per hundred acres from the field, where no two selected plants are within a set distance of each other;
(b) analyzing each selected plant's root system, where said analyzing comprises determining the if the damage to the plant root mass is at, above or below a predetermined score; and
(c) calculating a composite root damage score for the field based on the number of plants with scores at or above the predetermined score.
